(19) United States
(12) Patent Application Publication
(10) Pub. No.: US 2022/0354973 A1
(43) Pub. Date: Nov. 10, 2022

(54) IRON NITRIDE NANOPARTICLE SUSPENSION

(71) Applicant: Regents of the University of Minnesota, Minneapolis, MN (US)

(72) Inventors: Jian-Ping Wang, Shoreview, MN (US); Kai Wu, Saint Paul, MN (US); Bin Ma, St Paul, MN (US); Jinming Liu, Fremont, CA (US)

(21) Appl. No.: 17/659,953

(22) Filed: Apr. 20, 2022

Related U.S. Application Data
(60) Provisional application No. 63/201,246, filed on Apr. 20, 2021.

(51) Int. Cl. A61K 49/18; C09C 1/22; C09C 3/00; C09C 3/04; C09C 3/08; C09C 3/10

(52) U.S. Cl. CPC A61K 49/1854; A61K 49/186; C09C 1/22; C09C 3/006; C09C 3/041; C09C 3/08; C09C 3/10; C01P 2004/64; C01P 2006/42; B82Y 5/00

(57) ABSTRACT

A method may include wet ball milling a plurality of iron nitride nanoparticles in the presence of a surface active agent to modify a surface of the plurality of iron nitride nanoparticles and form a plurality of surface-modified iron nitride nanoparticles for a variety of biomedical applications and soft magnetic materials related applications.

This application claims the benefit of U.S. Provisional Patent Application No. 63/201,246, filed Apr. 20, 2021, the entire contents of which is incorporated herein by reference.

### TECHNICAL FIELD

The disclosure relates to magnetic nanoparticles and techniques for forming magnetic nanoparticles.

### BACKGROUND

Iron oxide nanoparticles (IONPs) have been widely used for magnetic diagnosis and treatment such as magnetic resonance imaging (MRI), magnetic particle imaging (MPI), gene/drug delivery, magnetic hyperthermia therapy, magnetic separation, and magnetic biosensors. The research interest in pursuing inexpensive, high magnetic moment, low biotoxicity, colloidal stable, and environmentally friendly magnetic nanoparticles (MNPs) is growing rapidly in view of the increasing demands of high sensitivity magnetic diagnosis and low dose treatments. To this end, γ′-Fe₄N is one promising candidate, because 1) both iron and nitrogen are inexpensive, low biotoxicity, and environment friendly; and 2) the saturation magnetization of γ′-Fe₄N is around two times higher than that of IONPs. Minnealloy α″-Fe₁₆NₓZ₂₋ₓ or α′-Fe₈NₓZ₁₋ₓ) is another promising candidate, because of its giant saturation magnetization and ultralow magnetic anisotropy constant.

### SUMMARY

In some examples, the disclosure describes a method that includes wet ball milling a plurality of iron nitride nanoparticles in the presence of a surface active agent to modify a surface of the plurality of iron nitride nanoparticles and form a plurality of surface-modified iron nitride nanoparticles.

In some examples, the disclosure describes a suspension that includes a solvent and a plurality of surface-modified iron nitride nanoparticles suspended in the solvent.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 is a flow diagram illustrating an example technique for forming magnetic nanoparticles that include iron nitride (e.g., γ′-Fe₄N, γ′-Fe₄NₓZ₁₋ₓ, α′-Fe₈N, α″-Fe₁₆NₓZ₂₋ₓ, or α′-Fe₈NₓZ₁₋ₓ, wherein Z comprises at least one of C, B, P, Si, or O).

FIG. 2A is a conceptual diagram of an example tube furnace.

FIG. 2B is a series of example space-filling models of γ-Fe₂O₃, Fe, and γ′-Fe₄N crystal structure transitions from reduction and nitridation steps.

FIG. 2C shows example space-filling models of nitrogen atoms on the surface of an Fe nanoparticle.

FIGS. 3A-3E show schematic views of example γ′-Fe₄N nanoparticle surface modification steps.

FIG. 4 illustrates a series of photographs of the samples at various stages of FIGS. 3A-3E.

FIGS. 5A and 5B are static magnetic hysteresis loops of γ-Fe₂O₃ nanoparticles and synthesized γ′-Fe₄N nanoparticles.

FIG. 5C shows XRD patterns of γ-Fe₂O₃ nanoparticles and synthesized γ′-Fe₄N nanoparticles.

FIG. 5D shows ball-and-stick models of γ-Fe₂O₃, Fe, and γ′-Fe₄N.

FIG. 6 shows a series of TEM images of X@2k and X@11k samples (X=A, B, C, and D) under different scale bars.

FIGS. 7A-7C are plots of magnetic core size distributions of γ′-Fe₄N nanoparticles from samples X@11k obtained from the TEM images.

FIGS. 8A-8D are plots showing the hydrodynamic size distributions of X@2k samples, X=A, B, C, and D, respectively, measured by NTA.

FIGS. 8E-8H are NTA photos of γ′-Fe₄N nanoparticles from X@2k samples.

FIGS. 9A-9D are plots showing the hydrodynamic size distributions of X@11k samples, X=A, B, C, and D, respectively, measured by NTA.

FIGS. 9E-9H are NTA photos of γ′-Fe₄N nanoparticles from X@11k samples.

FIG. 10A shows a series of photos of suspensions including surface functionalized γ′-Fe₄N nanoparticles through a 21-day observation period.

FIGS. 10B-10F show magnetically separated γ′-Fe₄N nanoparticle wet cakes by a permanent magnet.

FIGS. 11A-11D illustrate IR transmittance spectrums of samples X@pellet.

FIGS. 11E-11G illustrate the chemical structures of original surface-active media.

FIGS. 12A-12C show schematic views of chemical groups bonding to Fe atoms through the hydroxyl groups on the surface of γ′-Fe₄N nanoparticles.

FIGS. 13A-13E illustrate crystal structure characterizations on synthesized γ′-Fe₄N nanoparticle powder and X@pellet samples.

### DETAILED DESCRIPTION

Iron nitride (such as γ′-Fe₄N, α′-Fe₈N, α″-Fe₁₆NₓZ₂₋ₓ, or α′-Fe₈NₓZ₁₋ₓ, wherein Z comprises at least one of C, B, or O) is one promising candidate for a material in magnetic nanoparticles, as 1) both iron and nitrogen are inexpensive, low biotoxicity, and environment friendly, and 2) the saturation magnetization of γ′-Fe₄N is around two times higher than that of IONPs. A comparison of bulk magnetic materials is shown below in Table 1.

TABLE 1
Comparison of Magnetic Properties of Different Bulk Materials

| | | | Critical size |
|---|---|---|---|
| | | | (diameter) to be |
| | | | superparamagnetic |
| Material | Mₛ (emu/g) | K (erg/cm³) | (nm) |
| Fe₃O₄ | 98 | −1.1 × 10⁵ | 26 |
| γ-Fe₂O₃ | 82 | −4.6 × 10⁴ | 35 |
| FeCo | 242 | 10⁵ | 27 |
| Fe | 221 | K₁ = 4.8 × 10⁵ | 16 |
| | | K₂ = ±0.5 × 10⁵ | |
| γ′-Fe₄N | 184 | 2.9 × 10⁵ | 19 |
| α″-Fe₁₆N₂ | 226 | 9.6 × 10⁶ | 5.9 |
| α″-Fe₁₆CN | 258 | 2.4 × 10⁶ | 9.4 |

In view of saturation magnetizations, iron nitride nanoparticles (γ′-Fe₄N, α′-Fe₈N, α″-Fe₈N₂₋ₓZₓ and α′-Fe₈N₁₋ₓZₓ nanoparticles, where Z includes at least one of C, B, or O) and FeCo nanoparticles show higher saturation magnetizations over iron oxide. However, FeCo cannot be directly used in biomedical applications because of the concerns of oxidation and corrosion. Thus, using FeCo requires a core-shell structure nanoparticle, with the high saturation FeCo magnetization material as the core and protective biocompatible nonmagnetic materials as the shell to prevent oxidation as well as improve biocompatibility. This reduces the overall saturation magnetization of the core-shell nanoparticles.

The preparation of colloidally stable, uniformly sized, and sub-100-nm γ′-Fe₄N nanoparticles for biomedical applications is challenging. A gas nitriding method has been used to mass production γ′-Fe₄N nanoparticles. In the gas nitriding method, IONPs may be used as a precursor and a hydrogen reduction step is carried out to remove oxygen and obtain reduced Fe nanoparticles. Next, a mixture of ammonia and hydrogen gas is used to nitride the Fe nanoparticles at a temperature between about 400° C. and about 600° C. Single-phase γ′-Fe₄N nanoparticles have been reported using this method. Although the high temperature gas nitriding approach can realize massive production of high-magnetic-moment γ′-Fe₄N nanoparticles, due to the relatively high processing temperature, some nanoparticles are sintered together, leading to a larger and wider size distribution of the resulting material. The sintered, larger nanoparticles show remanent magnetization (i.e., are not superparamagnetic, showing magnetizations at zero external field). This type of nanoparticles will agglomerate and may block the blood vessels for in vivo applications (such as hyperthermia therapy) and will cause non-uniform magnetic signals for in vitro bioassays. Such material may be less useful, as nanoparticles that are sub-100-nm and with a narrow size distribution are required for most biomedical applications. Further, gas nitriding produces dry γ′-Fe₄N nanoparticles, in which weak forces such as electrostatics, van der Walls forces, and capillary effects may reduce stability of colloidal solutions of the γ′-Fe₄N nanoparticles. Although there have been many reports of the stabilization of IONPs with different functional groups in various solution conditions, direct surface chemical modification of γ′-Fe₄N nanoparticles has not been reported. This disclosure describes example, synthesis and surface functionalization strategies for obtaining stable, substantially monodispersed, sub-100-nm, narrow-size-distributed γ′-Fe₄N nanoparticle colloidal suspensions.

This disclosure describes synthesis methods for γ′-Fe₄N nanoparticles through a gas nitriding approach. These γ′-Fe₄N nanoparticles are then surface functionalized using wet ball milling and a surface-active media such as a commercially available nanoparticle surfactant, oleic acid (OA), or tetramethylammonium hydroxide solution (TMAOH). The surface-active nanoparticles then may be separated using a centrifugation process to collect the uniformly sized, sub-100-nm γ′-Fe₄N nanoparticles from the supernatants. The morphologies and hydrodynamic sizes of processed γ′-Fe₄N nanoparticles, as well as the surface chemical groups on nanoparticles were characterized by the standard transmission electron microscope (TEM), nanoparticle tracking analyzer (NTA), and Fourier-transform infrared spectroscopy (FTIR). The colloidal stability of processed γ′-Fe₄N nanoparticle suspensions was evaluated by a zeta potential analyzer (ZPA) and through a 21-day period observation period.

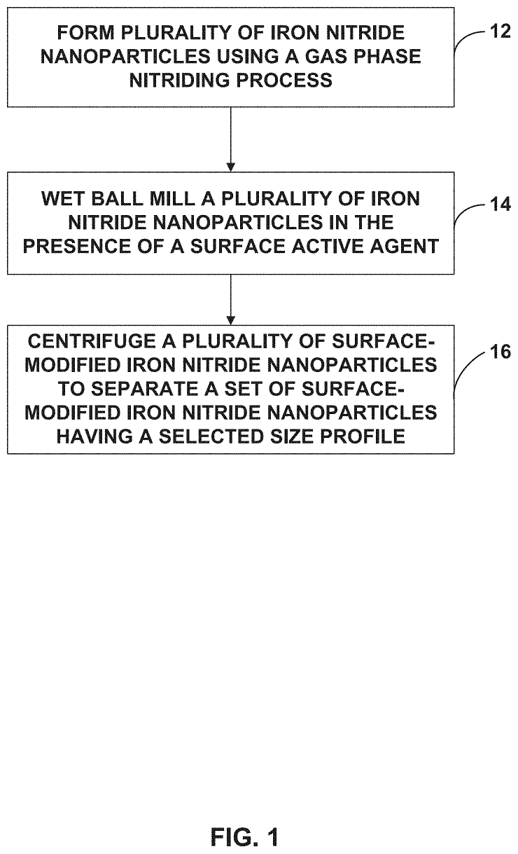

FIG. 1 is a flow diagram illustrating an example technique for forming magnetic nanoparticles that include iron nitride (e.g., γ′-Fe₄N, α′-Fe₈N, α″-Fe₁₆NₓZ₂₋ₓ, or α′-Fe₈NₓZ₁₋ₓ, wherein Z comprises at least one of C, B, or O). The technique of FIG. 1 optionally includes forming a plurality of iron nitride nanoparticles using a gas phase nitriding process (12). In some examples, the technique of FIG. 1 may omit step (12) and may begin with the following step.

In examples in which a plurality of iron nitride nanoparticles are formed using a gas phase nitriding process (12), the gas phase nitriding process may include placing a plurality of precursor nanoparticles in a furnace. The precursor nanoparticles may include iron oxide (e.g., γ-Fe₂O₃) nanoparticles, iron (e.g., elemental Fe) nanoparticles, or other iron-containing nanoparticles (e.g., iron carbide, iron boride, or the like). The furnace may include any suitable furnace, including a tube furnace.

In some examples in which the precursor nanoparticles include iron oxide, the gas phase nitriding process may include reducing the plurality of iron oxide nanoparticles to form a plurality of iron nanoparticles. In examples in which the precursor nanoparticles do not include iron oxide (e.g., examples in which the precursor nanoparticles include elemental iron, iron carbide, or iron boride), the reducing step may be omitted. The precursor nanoparticles may be reduced by introducing a reducing gas into the furnace. The reducing gas may include, for example, hydrogen gas, alone or mixed with another gas. In some examples, the reducing step may occur at a temperature of between about 350° C. and about 450° C. and a time between about 2 hours and about 5 hours.

Once the optional reduction step is complete, the resulting nanoparticles may include iron, iron carbide, iron boride, or the like. The nanoparticles then may be nitride. The nitriding may be performed by exposing the nanoparticles to elemental nitrogen or a nitrogen-containing compound, such as ammonia, urea, diatomic nitrogen, ammonium nitride, an amide, hydrazine, or the like. The nitriding may be performed at a temperature of between about 350° C. and about 450° C. (such as about 400° C.) and a time between about 2 hours and about 3 hours.

In some examples, during the nitriding step additional elements may be introduced to the nanoparticles. For example, at least one of carbon, boron, phosphorus, silicon, or oxygen may be introduces to the nanoparticles. Such elements may result in the formation of Fe₄N, Fe₄NₓZ₁₋ₓ, α″-Fe₁₆N₂₋ₓZₓ and/or α′-Fe₈N₁₋ₓZₓ, where Z includes at least one of C, B, P, Si, or O. Sources of carbon may include carbon monoxide, carbon dioxide, methane, graphite, urea, or the like. Some compounds may be used as a source of both carbon and nitrogen, such as urea.

The nitriding may form nanoparticles including iron nitride. The iron nitride may include at least one of γ′-Fe₄N, γ′-Fe₄NₓZ₁₋ₓ, α′-Fe₈N, α″-Fe₁₆NₓZ₂₋ₓ, or α′-Fe₈NₓZ₁₋ₓ, wherein Z comprises at least one of C, B, P, Si, or O. In some examples, the nanoparticles may include a mixture of iron nitride phases, may include iron nitride and one or more iron phases, or the like.

Regardless of whether the iron nitride is formed (12) or purchase, the technique of FIG. 1 then includes wet ball milling a plurality of iron nitride nanoparticles in the presence of a surface active agent to modify a surface of the plurality of iron nitride nanoparticles and form a plurality of surface-modified iron nitride nanoparticles (14). The iron nitride nanoparticles may include any of those described above, including at least one of γ′-Fe₄N, γ′-Fe₄NₓZ₁₋ₓ, α′-Fe₈N, α″-Fe₁₆NₓZ₂₋ₓ, or α′-Fe₈NₓZ₁₋ₓ, wherein Z comprises at least one of C, B, P, Si, or O. The surface-modifying agent may include any agent that reacts with a surface of the iron nitride nanoparticles to form a surface layer on the iron nitride nanoparticles. For example, the surface-modifying agent include at least one hydroxyl group that reacts with a surface of the iron nitride nanoparticles to form a surface layer on the iron nitride nanoparticles. Example surface-modifying agents include sources of at least one of polyvinylpyrrolidone, a polyoxyalkylene amine derivative, polyethylene glycol, ethylene glycol monobutyl ether, nonylphenol, or tetramethylammonium hydroxide.

The wet ball milling may be performed for between about 1 hour and about 10 hours at an rpm of between about 100 rpm and about 1000 rpm. The wet ball milling may help break up any sintered or agglomerated nanoparticle clumps while the surface modification reaction is proceeding.

The technique of FIG. 1 then optionally includes centrifuging the plurality of surface-modified iron nitride nanoparticles to separate a set of surface-modified iron nitride nanoparticles having a selected size profile from the plurality of surface-modified iron nitride nanoparticles (16). In some examples, the plurality of surface-modified iron nitride nanoparticles may be centrifuged at an rpm and for a time selected to obtain a set of surface-modified iron nitride nanoparticles having an average diameter of less than about 100 nm, or less than about 50 nm. In some implementations, the plurality of surface-modified iron nitride nanoparticles may be centrifuged at an rpm and for a time selected to obtain a set of surface-modified iron nitride nanoparticles having a substantially unimodal size distribution (e.g., at least 90% of the nanoparticles in the set have a size distributed around a single peak). In some examples, centrifuging the plurality of surface-modified iron nitride nanoparticles may be performed at an rpm between about 1,000 rpm and about 50,000 rpm, such as about 2,000 rpm or about 11,000 rpm.

The technique of FIG. 1 may optionally include further processing the surface-modified iron nitride nanoparticles into one or more mixtures or bulk materials. As one example, the surface-modified iron nitride nanoparticles may be selected for a relatively low average diameter and substantially unimodal size distribution and dispersed into a carrier fluid for use as a biomedical or therapeutic agent. As another example, the surface-modified iron nitride nanoparticles may have a wide distribution of hydrodynamic sizes due to surface functionalization and/or irregular shape, and may be selected to produce a mixture having a corresponding high magnetic field gradient.

In some examples, the surface-modified iron nitride nanoparticles may be consolidated into a bulk material, such as a soft magnet material. As one example, the surface-modified iron nitride nanoparticles may be dried and compacted into a dense bulk material having grain sizes that correspond to an average diameter of the surface-modified iron nitride nanoparticles. As another example, the surface-modified iron nitride nanoparticles may be dried and dispersed in a binder to form a composite material. The consolidated surface-modified iron nitride nanoparticles may have an irregular shape due to the forces exerted on the iron nitride nanoparticles during wet ball milling, and may be reflected by a high hydrodynamic size, surface area to volume ratio, or other measure of irregularity of a shape or surface.

Iron nitride nanoparticles produced using the techniques described herein may be used in a variety of applications in which high uniformity, high magnetic saturation, and/or low coercivity may be desired. In some examples, iron nitride nanoparticles may be used in biomedical applications including, but not limited to, magnetic resonance imaging (MRI), magnetic particle imaging (MPI), gene/drug delivery, magnetic hyperthermia therapy, magnetic separation, magnetic biosensors, and the like. As one example, a suspension of iron nitride nanoparticles surface functionalized through wet milling and subsequently centrifuged may have a relatively low size (e.g., an average diameter of less than about 20 nm) and high size uniformity (e.g., a 90% size distribution around a single peak) of the iron nitride nanoparticles. As another example, iron nitride nanoparticles formed by wet milling may have an irregular shape that produces a wide distribution of hydrodynamic sizes, resulting in increased transverse relaxivity. As a result, an MRI contrast agent that includes irregularly shaped iron nitride nanoparticles may have increased contrast.

In some examples, iron nitride nanoparticles may be used in soft magnet applications including, but not limited to, inductors, transformers, generators, motors, and the like. For example, iron nitride nanoparticles may be processed into a material having a high saturation magnetization (e.g., >180 emu/g) and low coercivity. Iron nitride nanoparticles having a sub-100 nm average diameter may be compacted or composited, alone or with a binder, to form a dense soft magnetic material. This soft magnetic material may have a relatively small grain size (e.g., <100 nm) corresponding to the small average diameter of the iron nitride nanoparticles, resulting in relatively low coercivity. The soft magnetic material may be incorporated into power electronics and other high temperature, high magnetic field devices.

### EXAMPLES

The γ-Fe₂O₃ nanoparticle powder was purchased from MTI Corporation. Commercial nanoparticle surfactant (in water dispersion) was purchased from US Research Nanomaterials, Inc. This surfactant product is a mixture of following substances: Nonylphenol (C₁₅H₂₄O, CAS#: 25154-52-3), Polyoxyalkylene amine derivative (CAS#: 68511-96-6), polyethylene glycol (C₁₄H₂₂O(C₂H₄O)ₙ, n=9-10, CAS#: 9002-93-1), Polyvinylpyrrolidone ((C₆H₉NO)ₙ, CAS#: 9003-39-8), Butyl ethanoate (C₆H₁₂O₂, CAS#: 123-86-4), Ethylene glycol monobutyl ether (C₆H₁₄O₂, CAS#: 111-76-2). Oleic acid (C₁₈H₃₄O₂, CAS#: 112-80-1) was purchased from Thermo Fisher Scientific. Tetramethylammonium hydroxide solution (N(CH₃)₄OH, CAS#: 75-59-225, wt. % in water) was purchased from Sigma Aldrich.

### Synthesis of γ′-Fe₄N Nanoparticles

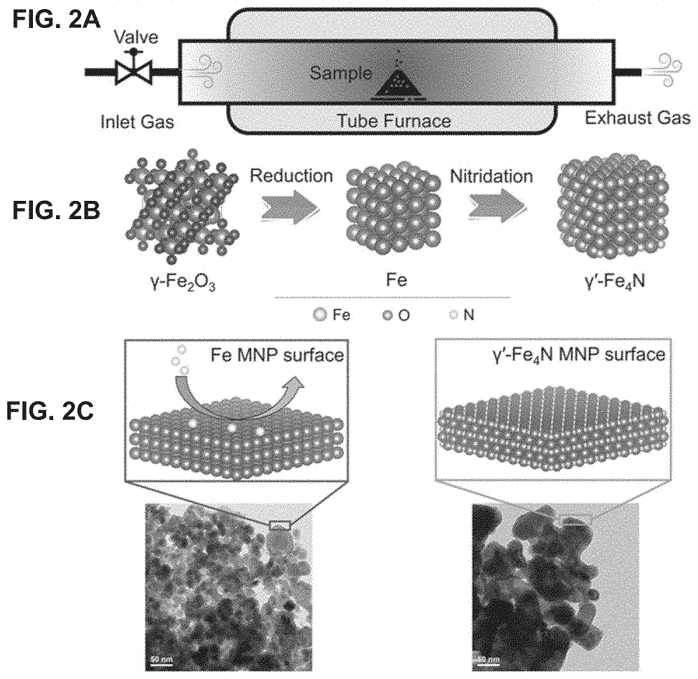

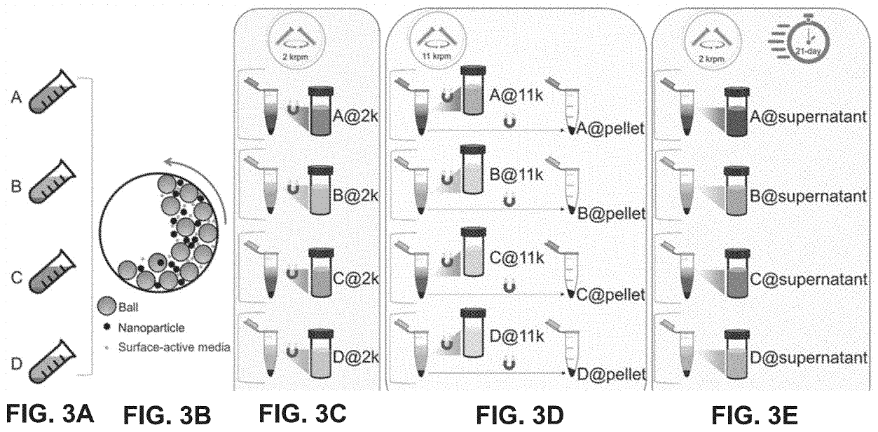

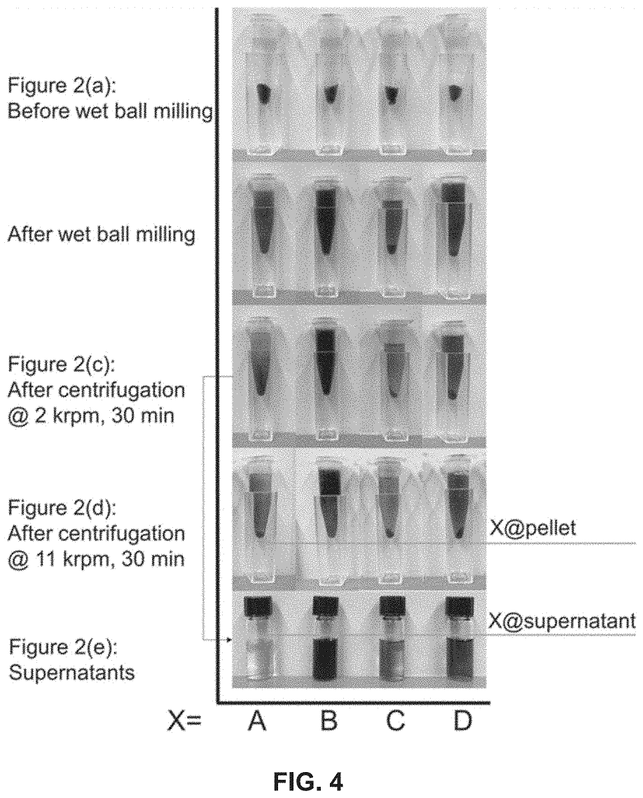

γ-Fe₂O₃ nanoparticles (nominal diameter of about 20 nm) were used to prepare γ′-Fe₄N by a gas nitriding approach. The γ-Fe₂O₃ nanoparticles were put in a tube furnace with a diameter of 1 inch, as shown in FIG. 2A. Once the 100 sccm H₂ gas was introduced, the furnace temperature was increased from room temperature to between about 350° C. and about 450° C. at a rate of about 10° C./min. The γ-Fe₂O₃ nanoparticles were reduced in hydrogen for between about 2 hours and about 5 hours to obtain Fe nanoparticles. This process corresponds to the reduction step labelled in FIG. 2B. Once the reduction was complete, the temperature of the tube furnace was kept at about 400° C. and ammonia at a rate of 60 sccm (or 40 sccm) and hydrogen at a rate of 20 sccm (or 40 sccm) were introduced into the tube furnace for nitriding as labelled in FIG. 2B. FIG. 2B is a series of space-filling models of γ-Fe₂O₃, Fe, and γ′-Fe₄N crystal structure transitions from reduction and nitridation steps.

The nitriding was performed for between 2 hours and 3 hours. Active nitrogen reacts with the reduced Fe to form γ′-Fe₄N, which is shown in the schematic drawing in FIG. 2C, which shows space-filling models of nitrogen atoms on the surface of an Fe nanoparticle. As shown in FIG. 2C, nitrogen atoms diffuse into reduced Fe nanoparticles to form γ′-Fe₄N. The insets shown in FIG. 2C are the TEM images of Fe and γ′-Fe₄N nanoparticles. After the nitriding was complete, the tube furnace was cooled to 50° C., then the γ′-Fe₄N nanoparticles were directly transferred to a glove box under a nitrogen atmosphere. The samples for vibrating sample magnetometer (VSM) and X-ray diffraction (XRD) were also prepared in the glove box to avoid further oxidation.

### γ′-Fe₄N Nanoparticles Surface Modification

Four γ′-Fe₄N nanoparticle suspensions were prepared through a wet ball milling and centrifugation process. FIGS. 3A-3E show schematic views of γ′-Fe₄N nanoparticle surface modification steps. To begin with, four samples of γ′-Fe₄N nanoparticle powder in surface-active media were prepared. Sample A included 50 mg of γ′-Fe₄N nanoparticle powder was dispersed in 5 mL water. Sample B included 50 mg of γ′-Fe₄N nanoparticle powder dispersed in 5 mL of a 5 vol/vol % commercial nanoparticle surfactant in water. Sample C included 50 mg of γ′-Fe₄N nanoparticle powder dispersed in 5 mL of a 25 vol/vol % oleic acid (OA) in ethanol solution. Sample D included 50 mg of γ′-Fe₄N nanoparticle powder dispersed in 5 mL of a 25 wt. % tetramethylammonium hydroxide (TMAOH) in water solution, as shown in FIG. 3A. Each sample was then wet ball milled at 350 rpm for 4 hours with time intervals of 30 min, as shown in FIG. 3B.

Despite the high yield in the synthesis of γ′-Fe₄N nanoparticles by means of the gas nitriding approach, due the high temperatures in the gas nitriding process, the sintered masses of nanoparticles with large sizes are not favored for biomedical applications. Thus, a centrifugation step was carried out to maintain the sub-100 nm γ′-Fe₄N nanoparticles and discard larger nanoparticles. There is a trade-off between obtaining the uniformly sized, sub-100 nm nanoparticles and reducing the loss rate of raw materials (i.e., the nanoparticle powder after the wet ball milling step). Two centrifugal speeds, 2,000 rpm and 11,000 rpm, were used to determine the effect of centrifugal speeds on yield and particle size distribution. More nanoparticles were retained, and the particle size distribution was wider when sample was centrifuged at 2,000 rpm. On the other hand, centrifugation at 11,000 rpm resulted in a narrower size distribution, but the number of nanoparticles recovered was lower.

After the wet ball milling process, the turbid suspensions for each sample were allotted into three 1.5 mL vials. The first vial was centrifuged at 2,000 rpm for 30 min, and a 500 μL of supernatant was drawn from the vial. The surface-functionalized γ′-Fe₄N nanoparticles from the supernatant of each sample was separated using a permanent magnet, then washed three times by either water (for nanoparticles from samples A, B, and D) or ethanol (for nanoparticles from sample C). Finally, the surface-functionalized γ′-Fe₄N nanoparticles from the supernatant of each sample were re-dispersed in water or ethanol solvent to the desired volumes for different characterization purposes. These γ′-Fe₄N nanoparticle suspensions were labeled as X@2k (X=A, B, C, and D), as shown in FIG. 3C.

Similarly, the second vial of each sample was centrifuged at 11,000 rpm for 30 min, and 500 μL of supernatant was drawn from the vial. After magnetic separation and wash out (as described above), the surface-functionalized γ′-Fe₄N nanoparticles from the supernatant of each sample were re-dispersed in water or ethanol solvent. These γ′-Fe₄N nanoparticle suspensions were labeled as X@11k (X=A, B, C, and D), as shown in FIG. 3D. The remaining wet cakes from the bottoms of these vials were magnetically separated, washed three times as described above, dried in a N₂ glove box at room temperature, and labeled as X@pellet (X=A, B, C, and D) for further characterizations.

The third vial of each sample was centrifugated at 2,000 rpm for 30 min, and a 1 mL of supernatant was drawn from the vial. These supernatants were labeled as X@supernatant (X=A, B, C, and D) and sealed in transparent glass bottles for a 21-day colloidal stability observation, as shown in FIG. 3E. FIG. 4 illustrates a series of photographs of the samples at various stages of FIGS. 3A-3E.

### Magnetic Property Characterization

Nanoparticles samples, γ-Fe₂O₃ and γ′-Fe₄N were stored in a N₂ glove box to avoid oxidation. All the nanoparticle samples for magnetic characterization were prepared in the N₂ glove box. Certain amounts of nanoparticles were weighed and sealed in parafilms to prevent further oxidation after the samples were taken out of the N₂ glove box. Hysteresis loops were measured by a Physical Properties Measurement System (PPMS, Quantum Design Inc.) to obtain the magnetic properties of these nanoparticles such as the saturation (Mₛ) and coercivity (H_{c}).

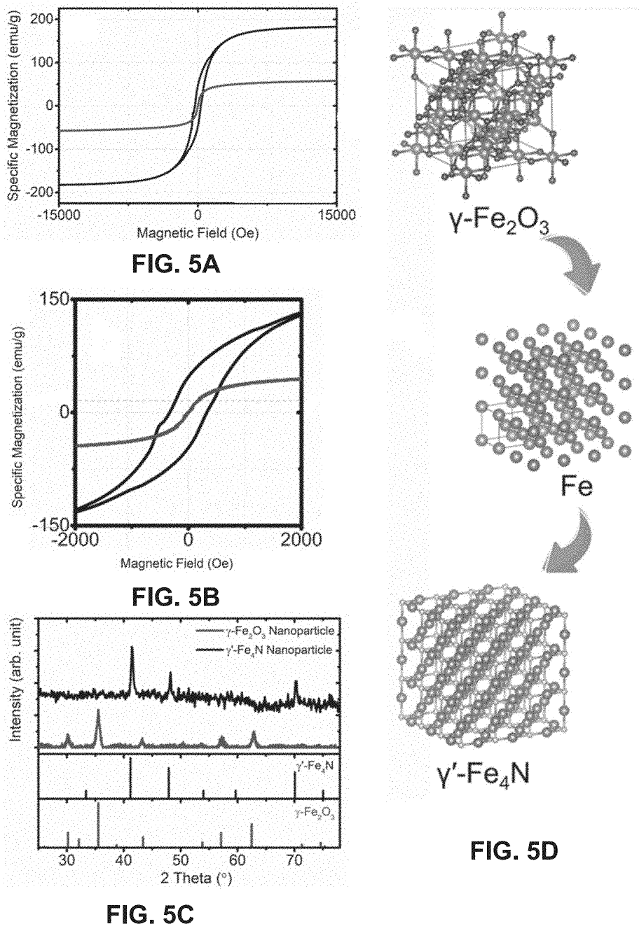

The static magnetic hysteresis loops of γ-Fe₂O₃ (the precursor) and the synthesized γ′-Fe₄N nanoparticles are shown in FIGS. 5A and 5B. As shown in FIG. 5A, the magnetization of γ-Fe₂O₃ and γ′-Fe₄N nanoparticles saturate at around 2.5 kOe and 7 kOe, respectively. The calculated specific saturation magnetizations (σₛ) of γ-Fe₂O₃ and synthesized γ′-Fe₄N nanoparticles at 15 kOe are about 57.7 and 182.7 emu/g, respectively. FIG. 5B is an enlarged view of the hysteresis loops within a magnetic field of ±2 kOe. The coercivities (H_{c}) of γ-Fe₂O₃ and synthesized γ′-Fe₄N nanoparticles are 0 and 310 Oe, respectively. Since some γ′-Fe₄N nanoparticles were sintered during the high temperature gas nitriding process, these sintered nanoparticles are larger in size thus, unlikely to be superparamagnetic. As a result, the synthesized γ′-Fe₄N nanoparticles show ferromagnetic properties with large magnetic coercivity and remanence as seen in FIG. 3A. On the other hand, the γ-Fe₂O₃ (the precursor) shows superparamagnetic properties with zero magnetic coercivity and remanence. The specific magnetizations (σ) of both nanoparticles under different magnetic fields, coercivities (H_{c}), remanence magnetizations (Mᵣ), and magnetic susceptibilities (χ) are summarized in Table 2.

TABLE 2
Magnetic properties comparisons on γ-Fe₂O₃ and
the synthesized γ′-Fe₄N nanoparticles.

| | Mₛ (15 kOe) | M (5 kOe) | M (1 kOe) | H_{c} | Mᵣ | χ (±1 kOe) |
|---|---|---|---|---|---|---|
| γ-Fe₂O₃ | 57.7 emu/g | 51.3 emu/g | 38.0 emu/g | 0 Oe | 0 emu/g | 0.038 emu/g/Oe |
| γ′-Fe₄N | 182.7 emu/g | 168.0 emu/g | 106.3 emu/g | 310 Oe | 45 emu/g | 0.093 emu/g/Oe |

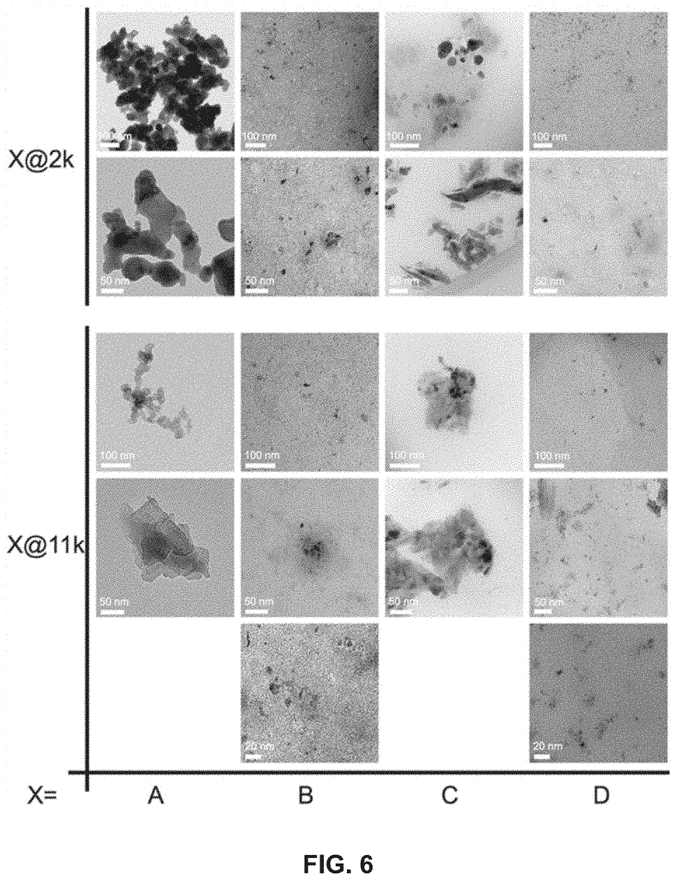

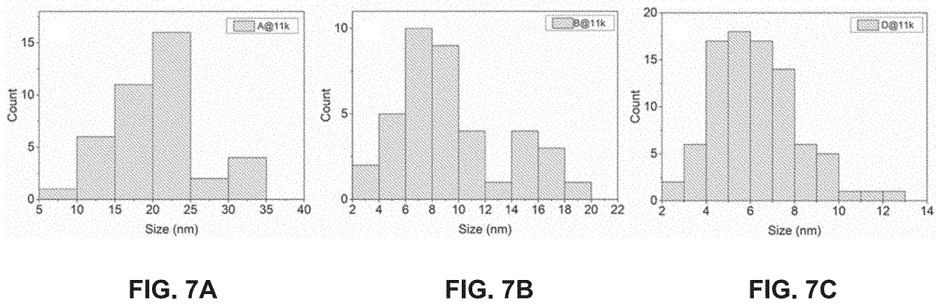

The high H_{c} and large remanence of γ′-Fe₄N are caused by the sintering during the high temperature gas nitriding process. These sintered γ′-Fe₄N particles with wider size distributions and larger sizes (as shown below in FIG. 6 and FIG. 7A) are not suitable for most biomedical applications. Thus, a goal of this disclosure is to break these larger sintered particles, separate smaller nanoparticles by a wet ball milling method with surface-active media, then centrifuge to extract and purify monodispersed, uniformly sized, and sub-100-nm γ′-Fe₄N nanoparticles.

### Crystal Structure Characterization

γ-Fe₂O₃ and γ′-Fe₄N nanoparticle samples for X-ray diffraction (XRD) patterns were also prepared in the N₂ glove box. Selected amounts of nanoparticles were put on a piece of glass substrate and an epoxy was used to seal the nanoparticles to prevent oxidation after the samples were taken out of the glove box. When the epoxy dried and solidified, these samples were used to for the XRD measurements. The XRD patterns were characterized using a Bruker D8 Discover 2D equipped with a Co radiation source operated at 45 kV and 40 mA. For a convenient comparison, the XRD patterns were converted into a Cu radiation source using MDI Jade software.

The XRD patterns of the precursor γ-Fe₂O₃ and synthesized γ′-Fe₄N nanoparticles are shown in FIG. 5C. The powder diffraction files (PDFs) of these two materials are also plotted at the bottom panels of the XRD patterns. The XRD pattern of starting γ-Fe₂O₃ nanoparticles matches well with the PDF, indicating that the starting materials are γ-Fe₂O₃. These iron oxide nanoparticles are then reduced by hydrogen gas to get rid of oxygen and create preferable microstructures for the following nitriding process. After the nitriding, the XRD patterns show that the main phase is γ′-Fe₄N. Thus, γ′-Fe₄N nanoparticles are successfully synthesized by the gas nitriding approach. Ball-and-stick models of γ-Fe₂O₃ and γ′-Fe₄N are shown in FIG. 5D to provide a brief view how the crystal structure changes from the γ-Fe₂O₃ to reduced Fe and synthesized γ′-Fe₄N.

### Nanoparticle Morphology and Size Characterization

The morphologies of γ′-Fe₄N nanoparticle cores were characterized by a transmission electron microscope (TEM, FEI Tecnai T12). Briefly, 10 μL of X@2k and X@11k (X=A, B, C, and D) nanoparticle colloidal suspensions were dropped on TEM grids and air dried at room temperature before TEM imaging.

The hydrodynamic sizes of these surface functionalized γ′-Fe₄N nanoparticles were measured using a nanoparticle tracking analyzer (NTA, Nanosight LM-10). 1.5 mL of samples X@2k and X@11k (X=A, B, C and D) were used for the hydrodynamic size measurements on the NTA. The NTA used a 400 nm (near UV) laser to track the motion of γ′-Fe₄N nanoparticles suspended in solvents (i.e., water for samples A, B, D and ethanol for sample C) then calculated the size distributions for nanoparticles between 10 nm and 1 μm.

Initially, it should be noted that the TEM morphology imaging was used to observe the magnetic cores of synthesized γ′-Fe₄N nanoparticles, thus, the sizes read from TEM images are different from the hydrodynamic sizes read from NTA.

FIG. 6 shows a series of TEM images of X@2k and X@11k samples (X=A, B, C, and D) under different scale bars. Samples A and C show severe aggregations of γ′-Fe₄N nanoparticles, while samples B and D show monodispersed nanoparticles even after the centrifugation at 2,000 rpm for 30 min.

It can be clearly seen that γ′-Fe₄N nanoparticles that are wet ball milled in water (samples A@2k and A@11k) and in oleic acid (OA) (samples C@2k and C@11k) are forming large clusters. This indicates that: 1) wet ball milling in water and OA cannot effectively separate γ′-Fe₄N nanoparticles; 2) these γ′-Fe₄N nanoparticles are not superparamagnetic and thus, forming clusters due to non-zero remanence; and 3) these γ′-Fe₄N nanoparticles processed with water and OA will not be colloidally stable and may form sediments in solvents.

On the other hand, the γ′-Fe₄N nanoparticles that are wet ball milled in commercial surfactant product and TMAOH are monodispersed. Samples B@2k, B@11k, D@2k, and D@11k show monodispersed and small size γ′-Fe₄N nanoparticles. This indicates that 1) centrifuging at 2,000 rpm for 30 min is sufficient to extract uniform-sized, monodispersed, and sub-100-nm γ′-Fe₄N nanoparticles; and 2) these nanoparticles are not forming clusters and will be colloidally stable. Furthermore, many γ′-Fe₄N nanoparticles with sizes below 20 nm are observed, while the theoretical size limit for γ′-Fe₄N nanoparticles to be superparamagnetic is 19 nm.

The magnetic core size distributions of γ′-Fe₄N nanoparticles from samples X@11k were obtained from the TEM images and plotted in FIGS. 7A-7C. Note that sample C@11k shows severe nanoparticle aggregations, and the size distribution was not clear from TEM images. The anisotropy constant of γ′-Fe₄N is reported to be K_{c}=2.9×10⁵ erg/cm³, and the theoretical critical size is calculated to be 19 nm for γ′-Fe₄N nanoparticles to be superparamagnetic at room temperature. Since water and oleic acid (OA) cannot effectively separate and surface functionalization γ′-Fe₄N nanoparticles, these nanoparticles are likely to cluster. As shown in FIG. 7A, a wide size distribution from 5 nm to 35 nm was obtained from TEM images for the sample ball milled in water. On the other hand, the sizes of γ′-Fe₄N nanoparticles from samples B@11k and D@11k were below 20 nm, as shown in FIGS. 7B and 7C. Thus, superparamagnetic γ′-Fe₄N nanoparticles were obtained from wet ball milling with commercial nanoparticle surfactants and TMAOH and centrifugation steps.

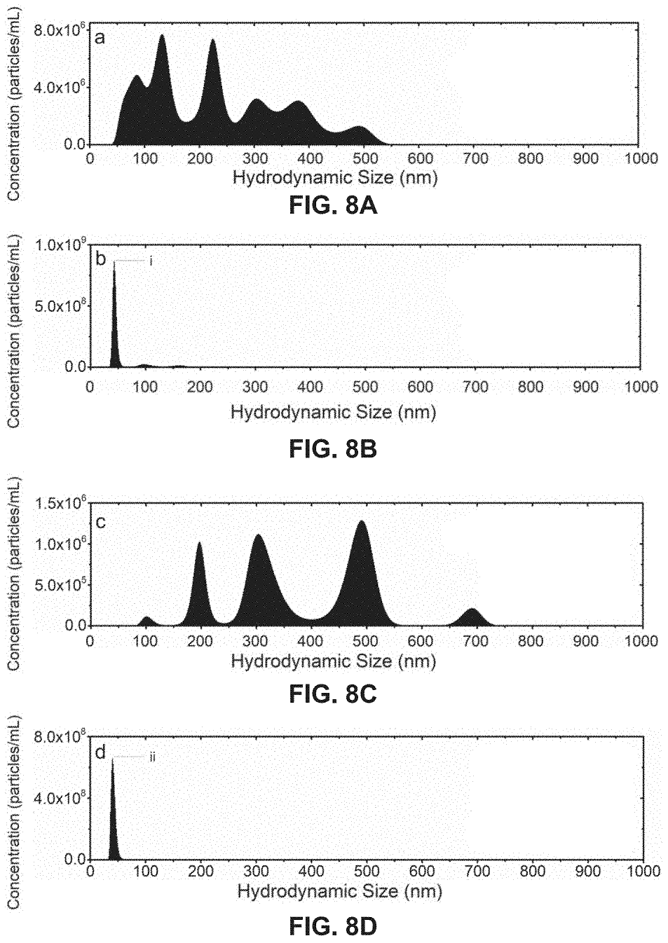

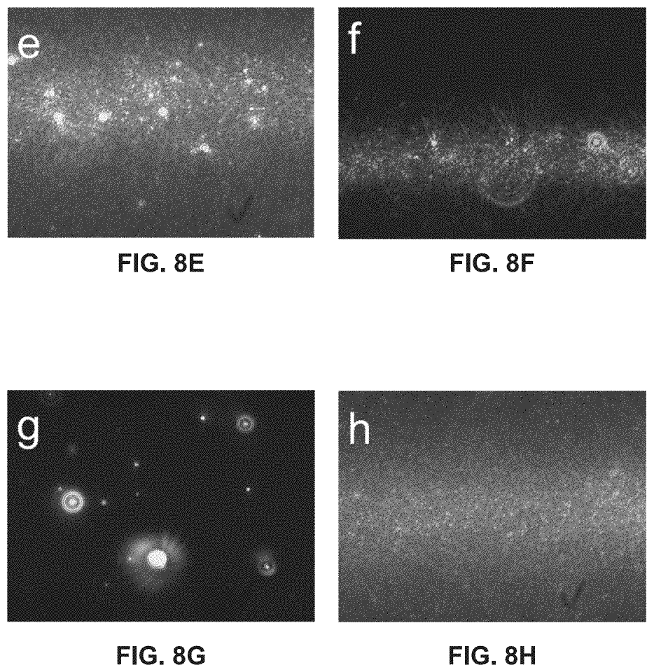

The NTA was used to measure the hydrodynamic size of the γ′-Fe₄N nanoparticles after different surface modification processes. FIGS. 8A-8D are plots showing the hydrodynamic size distributions of X@2k samples, X=A, B, C, and D, respectively, measured by NTA. FIGS. 8E-8H are the NTA photos of γ′-Fe₄N nanoparticles. (i) and (ii) denote the peaks at 44 nm and 41 nm, respectively. A statistical average of 5 independent NTA measurements was carried out on samples X@2k (X=A, B, C, and D). During each NTA measurement, a 1 min video of the γ′-Fe₄N nanoparticles is recorded under the microscope, and snapshots of the respective samples are given in FIGS. 8E-8H. From FIGS. 8A & 8E and FIGS. 8C & 8G, it is evident that the γ′-Fe₄N nanoparticles from samples A@2k and C@2k are severely clustered, with prominent peaks at the greater side of the hydrodynamic size. The significant clustering of the nanoparticles is also seen from the TEM images in FIG. 6 for samples A@2k and C@2k. However, from FIGS. 8B & 8F and FIGS. 8D & 8H, it is evident that the samples B@2k and D@2k show prominent peaks at hydrodynamic sizes of 44 nm and 41 nm, respectively. This is also supported by the TEM images in FIG. 6. The scale bars in TEM images of FIG. 6 show that some particles are of the order of 20 nm in size, whereas the hydrodynamic size measurements show that sample B@2k mostly has size of 44 nm and sample D@2k mostly has size of 41 nm because the surfactant coatings of nanoparticles sum up to the hydrodynamic size. On the contrary, TEM only captures the core size of the nanoparticles. Table 3 shows the 10^{th}, 50^{th} and 90^{th} percentile as well as the mean hydrodynamic sizes of γ′-Fe₄N nanoparticles from samples X@2k (X=A, B, C and D) as obtained from the NTA measurements.

TABLE 3
Percentile and average sizes of γ′-Fe₄N nanoparticles in X@2k
samples (X = A, B, C, and D).

| X@2k | A | B | C | D |
|---|---|---|---|---|
| 10^{th} | 86.8 nm | 39.7 nm | 198.1 nm | 37.8 nm |
| 50^{th} | 220.8 nm | 44.4 nm | 352.3 nm | 41.5 nm |
| 90^{th} | 405.3 nm | 102.8 nm | 514.7 nm | 49.8 nm |
| Average | 233.8 nm | 58.5 nm | 382.1 nm | 45.3 nm |
| size | | | | |

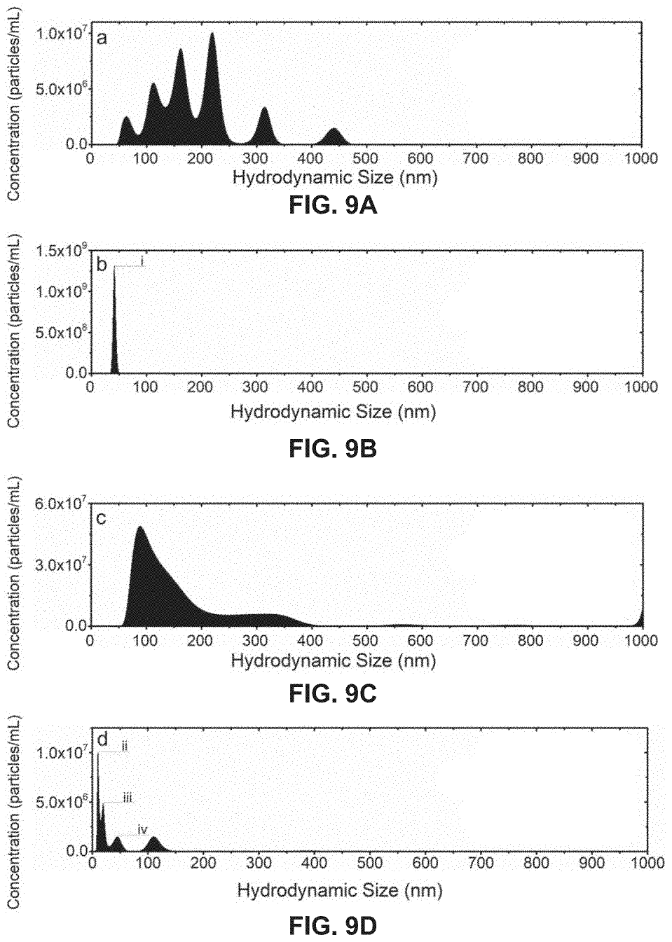

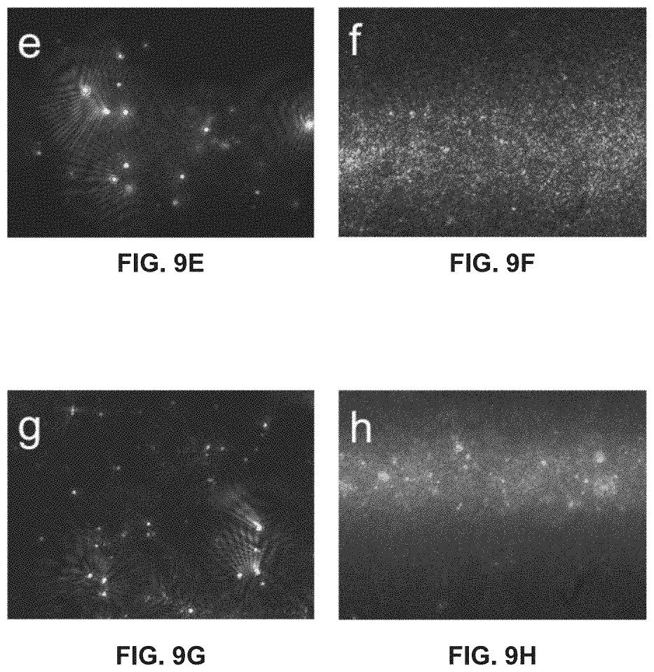

The hydrodynamic size distributions and percentile size distributions of samples X@11k (X=A, B, C, and D) are provided in FIGS. 9A-9H and Table 4.

From FIG. 9A in corroboration with snapshot in FIG. 9E and FIG. 9C in corroboration with snapshot in FIG. 9G, it is evident that the γ′-Fe₄N nanoparticles from samples A@11k and C@11k are severely clustering, with prominent peaks at the greater side of the hydrodynamic size. However, from FIG. 9B in corroboration with snapshot in FIG. 9F and FIG. 9D in corroboration with snapshot in FIG. 9H, it is evident that the sample B@11k shows prominent peak at 42 nm and sample D@11k shows prominent peaks at 11 nm, 20 nm, and 44 nm, respectively. Due to the ultra-high centrifugal speed, smaller nanoparticles are retained in the supernatants. Thus, 11 nm and 20 nm nanoparticles are dominating in sample D@11k. Table 3 shows the 10^{th}, 50^{th} and 90^{th} percentiles as well as the mean hydrodynamic sizes of nanoparticles from samples X@11k (X=A, B, C and D) as obtained from the NTA measurements.

### Colloidal Stability of γ′-Fe₄N Nanoparticles

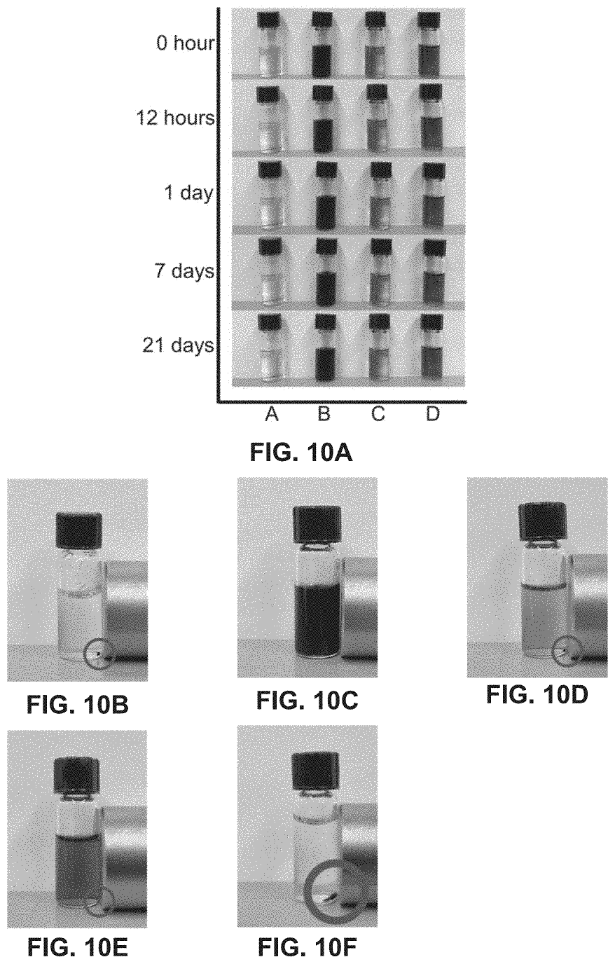

The colloidal stabilities of surface functionalized γ′-Fe₄N nanoparticles by different surface-active media were appraised through a 21-day observation period on samples X@supernatant (X=A, B, C, and D). As shown in FIG. 10A, no observable sedimentations are present in any of these samples. However, this type of observation by bare eyes are not very reliable due to the low amount of γ′-Fe₄N nanoparticles presenting in the suspensions. FIGS. 10B-10F show the magnetically separated γ′-Fe₄N nanoparticle wet cakes by a permanent magnet, proving that there is a small number of nanoparticles in each X@supernatant sample. It should be noted that due to the dark background color of solvent in sample B@supernatant, a second photo of the separated nanoparticles (wet cake) is given in FIG. 10F after three times of magnetic wash out with water.

### Zeta Potential of Nanoparticles

A zeta potential analyzer (ZPA, Stabino) was used to measure the particle charge distribution or the zeta potential of the surface functionalized γ′-Fe₄N nanoparticles from solvents (water or ethanol). 5 mL of samples X@2k (X=A, B, C, and D) are prepared, sonicated for 30 min before the zeta potential measurements.

The zeta potentials of surface functionalized γ′-Fe₄N nanoparticles in solvent are −29.13 mV, −26.27 mV, −0.55 mV, and −29.99 mV, respectively for A, B, C, and D. This indicates that γ′-Fe₄N nanoparticles processed by commercial surfactants, water, and TMAOH are relatively stable due to a large electrokinetic potential (or electrostatic repulsion), while, on the other hand, γ′-Fe₄N nanoparticles treated by OA will rapidly coagulate.

Although zeta potential results indicate γ′-Fe₄N nanoparticles treated by commercial surfactants, water, and TMAOH should be colloidally stable and that γ′-Fe₄N nanoparticles treated by OA should not be colloidally stable, the TEM images and NTA results show that only the γ′-Fe₄N nanoparticles that are treated by commercial surfactants and TMAOH are uniformly sized and monodispersed, while nanoparticles treated by water are not. This is because, although γ′-Fe₄N nanoparticles treated by water show a large electrokinetic potential (or electrostatic repulsion), there is no surface chemical groups functionalized on these nanoparticles, and the non-zero remanences of these nanoparticles cause clustering.

### Nanoparticle Surface Chemical Groups Characterization

The chemical groups functionalized on γ′-Fe₄N nanoparticle surfaces were characterized by a FTIR spectrometer (Fourier-transform infrared spectroscopy, Thermo Scientific Nicolet iS50 FTIR) on the main detector MCT-A (7000-600 cm⁻¹), with KBr beam splitter and a resolution of 2 cm⁻¹. A total of 32 scans were taken on each sample. Samples X@pellet (X=A, B, C, and D) were evenly spread and dried on a barium fluoride (BaF₂) window (diameter 25.4 mm, thickness 2 mm, purchased from EKSMA Optics, UAB) for FTIR transmittance spectrum collection. In addition, the FTIR transmittance spectrum of the commercial surfactant product, OA, and TMAOH are also collected for direct comparisons. These liquid samples were sealed between two BaF₂ windows before FTIR characterizations.

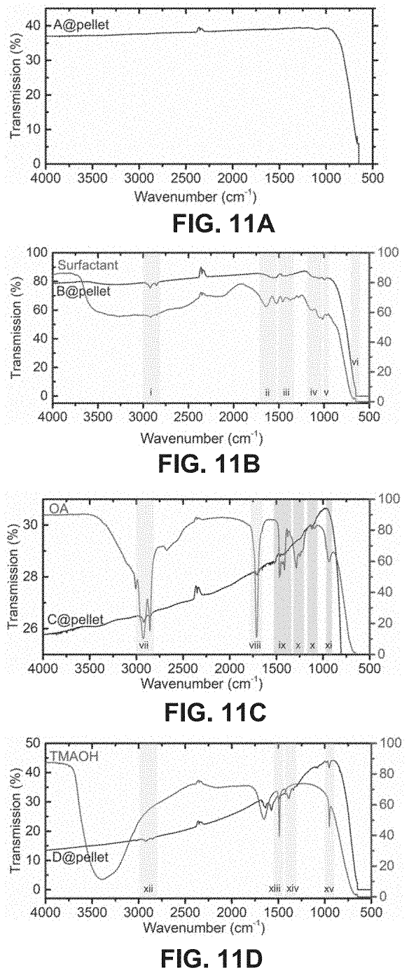

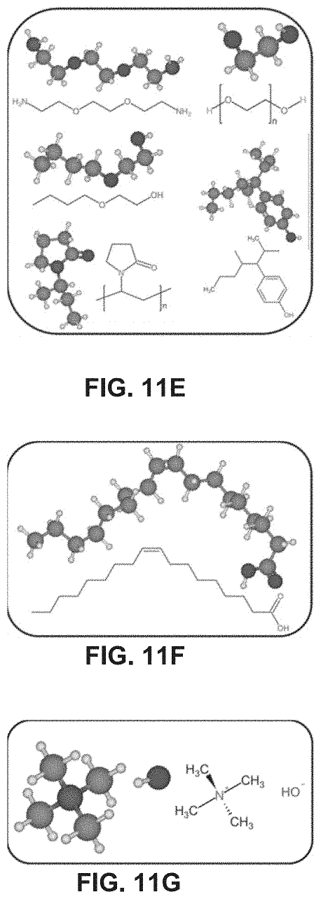

The IR transmittance spectrums are given in FIGS. 11A-11D. For comparison, the transmittance spectrums of original surface-active media (commercial surfactant for sample B, OA for sample C, TMAOH for sample D) are plotted in parallel. The grey regions indicate that the characteristic transmittance peaks are observed from both X@pellet and its original surface-active media. The ix, x, x, and xi regions in FIG. 11C indicate that the characteristic transmittance peaks from original surface-active media are absent from C@pellet sample. The chemical structures of original surface-active media are drawn in parallel with IT transmittance spectrums in FIGS. 11E-11G.

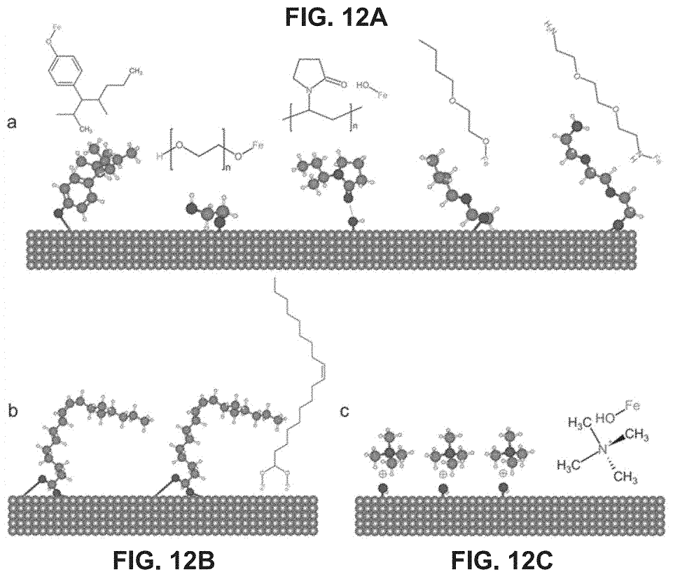

The FTIR transmittance spectrum of A@pellet in FIG. 11A does not show any remarkable peaks, thus, γ′-Fe₄N nanoparticles are not functionalized with any chemical groups through wet ball milling process in water. Note that, once the γ′-Fe₄N nanoparticles were brought out of the vacuum chamber, a mixed mode layer of hydroxyl group and water formed on the nanoparticle surface. This caused some weak spikes around 3600 cm⁻¹ and 1600 cm⁻¹. Usually, these peaks from water are removed by dehumidifying or pumping the FTIR chamber, subtracted as background spectrum (without sample loaded, only BaF₂ window loaded), and averaged by carrying out 32 FTIR scans. However, from sample C@pellet, as shown in FIG. 11C, some visible peaks caused by this mixed mode layer of hydroxyl group and water are still present. It does not affect the analysis of FTIR results. On the other hand, this mixed mode layer of hydroxyl group and water facilitates the subsequent nanoparticle surface modifications. FIGS. 12A-12C show schematic views of these chemical groups bonding to the Fe atoms through the hydroxyl groups on the surface of γ′-Fe₄N nanoparticles. The hydroxyl group on the surface of the nanoparticles tend to bond with the chemical groups with high electron affinity. The grain size, chemical structures of the final product can be engineered by optimizing the type and concentration of the surfactants.

In FIG. 11B, the chemical groups such as benzene derivative (˜700 cm⁻¹, marked as vi in FIG. 11B) and C═C bending (960-1000 cm⁻¹, marked as v in FIG. 11B) are from nonylphenol, C—N and C—O stretching (1000-1300 cm⁻¹, marked as vi in FIG. 11B) come from nonylphenol, polyoxyalkelene amine derivative, polyethylene glycol, polyvinylpyrrolidone, and ethylene glycol monobutyl ether. Other chemical groups and corresponding FTIR transmittance peaks are listed in Table 4. FTIF confirms that the surface-active groups from this commercial surfactant product are successfully functionalized on γ′-Fe₄N nanoparticles after the wet ball milling process. FIG. 12A gives the schematic views of these chemicals bond on γ′-Fe₄N nanoparticle surface through hydroxyl groups.

TABLE 4
Chemical groups coated on γ′-Fe₄N nanoparticles

| Sample | | | |
|---|---|---|---|
| index | Label | Chemical group | Sources |
| B@pellet | i | C—H stretching | All* |
| | | C═O stretching | Polyvinylpyrrolidone |
| | ii | C—O stretching | Polyoxyalkylene amine derivative, |
| | | | Polyethylene glycol, Ethylene glycol |
| | | | monobutyl ether |
| | | N—H bending | Polyoxyalkylene amine derivative |
| | iii | O—H bending | Nonylphenol, Polyethylene glycol, |
| | | | Ethylene glycol monobutyl ether |
| | | C—H bending | All* |
| | iv | C—N stretching | Polyoxyalkylene amine derivative, |
| | | | Polyvinylpyrrolidone |
| | | C—O stretching | Polyoxyalkylene amine derivative, |
| | | | Polyethylene glycol, Ethylene glycol |
| | | | monobutyl ether |
| | v | C═C bending | Nonylphenol |
| | vi | Benzene | Nonylphenol |
| | | derivative | |
| C@pellet | vii | C—H stretching | Oleic acid (OA) |
| | viii | C═O stretching | |
| | ix | C—H bending | |
| | x | C—O stretching | |
| | xi | C═C bending | |
| D@pellet | xii | C—H stretching | Tetramethylammonium hydroxide |
| | xiii | C—H bending | (TMAOH) |
| | xiv | C—N stretching | |
| | xv | C—H bending | |

*Nonylphenol, Polyoxyalkylene amine derivative, Polyethylene glycol, Polyvinylpyrrolidone, Ethylene glycol monobutyl ether

The transmittance spectrum of C@pellet is shown in FIG. 11C. The very weak peaks of C—H stretching (2800-3000 cm⁻¹, marked as vii in FIG. 11C) and C═O stretching (˜1750 cm⁻¹, marked as viii in FIG. 11C) were observed. However, the stronger C—H bending (1400-1500 cm⁻¹, marked as ix in FIG. 11C), C—O stretching (˜1250 cm⁻¹ and 1100 cm⁻¹, marked as x in FIG. 11C), C═C bending peak at 885-995 cm⁻¹, marked as xi in FIG. 11C) are absent from C@pellet. Thus, the OA is not successfully functionalized on γ′-Fe₄N nanoparticles. That also explains why these nanoparticles are clustering from TEM images (as shown in FIG. 6), showing wide and large size distributions (as shown in FIG. 7C), as well as low zeta potentials. Although the OA groups have been reported to be successfully coated on iron oxide nanoparticle by many groups, unfortunately, only very few amount of OA groups are bond on γ′-Fe₄N nanoparticles. FIG. 12B shows the schematic views of OA bond on γ′-Fe₄N nanoparticle surface.

The transmittance spectrum of D@pellet is shown in FIG. 11D. The characteristic C—H bending and stretching peaks at 2840-3000 cm⁻¹ (marked as xii in FIG. 11D), 1380-1450 cm⁻¹ (marked as xiii in FIG. 11D), and 880 cm⁻¹ (marked as xv in FIG. 11D), C—N stretching at 1300 cm⁻¹ (marked as xiv in FIG. 11D) are observed. This indicates that TMAOH has been successfully functionalized on γ′-Fe₄N nanoparticles. FIG. 12C shows the schematic views of TMAOH bond on γ′-Fe₄N nanoparticle surface.

### Crystal Structure of Surface Functionalized γ′-Fe₄N Nanoparticles

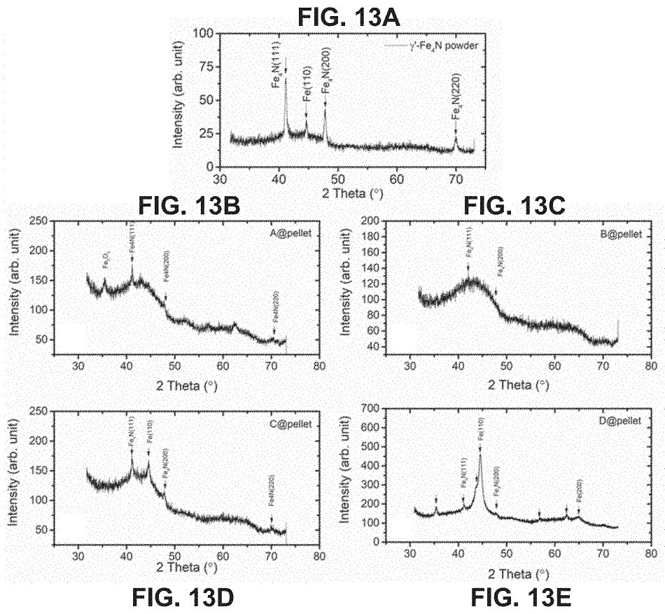

During the wet ball milling process, different surfactants were added, and the milling speed was low (350 rpm). The decomposition temperature of γ′-Fe₄N is above 600° C. based on the Fe—N phase diagram. The surfactants, except water, can also help protect nanoparticles from oxidation during the milling process. Thus, the produced nanoparticles should still conserve the original phase and have the similar magnetic properties. To demonstrate that the nanoparticles after wet ball milling with the four surface-active media (A: water, B: 5 vol/vol % commercial nanoparticle surfactant in water, C: 25 vol/vol % OA in ethanol, D: 25 wt % TMAOH in water) are still γ′-Fe₄N, a new batch of γ′-Fe₄N nanoparticles were synthesized and X@pellet samples (X=A, B, C, and D) were prepared as described above. The crystal structure characterizations on the newly synthesized γ′-Fe₄N nanoparticle powder and X@pellet samples are shown in FIGS. 13A-13E. The XRD patterns of newly synthesized γ′-Fe₄N nanoparticles and the γ′-Fe₄N peaks are labeled. However, for this batch of sample, reduced Fe phase is seen due to insufficient nitriding step from FIG. 13A. But this does not affect the assessment of whether wet ball milling with the four surface-active media will change the crystal structure of nanoparticles. FIGS. 13B-13E shows the XRD patterns of A@pellet, B@pellet, C@pellet, and D@pellet after the wet ball milling process. All the visible Fe₄N (111) and Fe₄N(200) peaks are labeled, as well as the Fe(110) peak (due to insufficient nitriding step, which is shown in the starting material γ′-Fe₄N powder). These figures indicate that the wet ball milling step with four surface-active media does not change the crystal structures of the produced nanoparticles. The crystal structure characterizations by XRD are strong evidence that the produced nanoparticles after ball milling step are still γ′-Fe₄N.

### Example 1

A method includes wet ball milling a plurality of iron nitride nanoparticles in the presence of a surface active agent to modify a surface of the plurality of iron nitride nanoparticles and form a plurality of surface-modified iron nitride nanoparticles.

### Example 2

The method of example 1, wherein the plurality of iron nitride nanoparticles comprise at least one of γ′-Fe₄N, α′-Fe₈N, α″-Fe₁₆NₓZ₂₋ₓ, or α′-Fe₈NₓZ₁₋ₓ, wherein Z comprises at least one of C, B, P, Si, or O.

### Example 3

The method of any of examples 1 and 2, further includes centrifuging the plurality of surface-modified iron nitride nanoparticles to separate a set of surface-modified iron nitride nanoparticles having a selected size profile from the plurality of surface-modified iron nitride nanoparticles.

### Example 4

The method of any of examples 1 through 3, further includes forming the plurality of iron nitride nanoparticles from a plurality of iron oxide nanoparticles using a gas phase nitriding process.

### Example 5

The method of example 4, wherein the plurality of iron oxide nanoparticles comprise a plurality of γ-Fe₂O₃ nanoparticles.

### Example 6

The method of any of examples 4 and 5, wherein the gas phase nitriding process comprises: reducing, in a furnace, the plurality of iron oxide nanoparticles to form a plurality of iron nanoparticles; and nitriding the plurality of iron particles to form the plurality of iron nitride nanoparticles.

### Example 7

The method of example 6, wherein nitriding the plurality of iron particles to form the plurality of iron nitride nanoparticles further comprises forming the plurality of iron nitride nanoparticles comprising at least one of Fe₄N, Fe₄NₓZ₁₋ₓ, α″-Fe₁₆NₓZ₂₋ₓ or α′-Fe₈NₓZ₁₋ₓ, where Z includes at least one of C, B, P, Si, or O based on introduction of at least one of carbon, boron, silicon, phosphorus, or oxygen to the furnace.

### Example 8

The method of any of examples 1 through 7, wherein wet ball milling the plurality of iron nitride nanoparticles in the presence of a surface active agent comprises wet ball milling the plurality of iron nitride nanoparticles in the presence of a source of at least one of: polyvinylpyrrolidone, a polyoxyalkylene amine derivative, polyethylene glycol, ethylene glycol monobutyl ether, nonylphenol, or tetramethylammonium hydroxide.

### Example 9

The method of any of examples 1 through 8, wherein wet ball milling the plurality of iron nitride nanoparticles in the presence of a surface active agent comprises wet ball milling for a period of between about 1 hour and about 10 hours at an rpm of between about 100 rpm and about 1000 rpm.

### Example 10

The method of any of examples 3 through 9, wherein centrifuging the plurality of surface-modified iron nitride nanoparticles comprises centrifuging the plurality of surface-modified iron nitride nanoparticles at an rpm and for a time selected to obtain a set of surface-modified iron nitride nanoparticles having an average diameter of less than about 100 nm.

### Example 11

The method of example 10, wherein centrifuging the plurality of surface-modified iron nitride nanoparticles comprises centrifuging the plurality of surface-modified iron nitride nanoparticles at a revolutions per minute (RPM) and for a time selected to obtain a set of surface-modified iron nitride nanoparticles having an average diameter of less than about 50 nm.

### Example 12

The method of any of examples 3 through 11, wherein centrifuging the plurality of surface-modified iron nitride nanoparticles comprises centrifuging the plurality of surface-modified iron nitride nanoparticles at an rpm and for a time selected to obtain a set of surface-modified iron nitride nanoparticles having unimodal size distribution.

### Example 13

The method of any of examples 3 through 12, wherein centrifuging the plurality of surface-modified iron nitride nanoparticles comprises centrifuging the plurality of surface-modified iron nitride nanoparticles at a revolutions per minute (RPM) between about 1,000 RPM and about 50,000 RPM.

### Example 14

The method of example 13, wherein centrifuging the plurality of surface-modified iron nitride nanoparticles comprises centrifuging the plurality of surface-modified iron nitride nanoparticles at an rpm of about 2,000 RPM or about 11,000 RPM.

### Example 15

A suspension includes a solvent; and a plurality of surface-modified iron nitride nanoparticles suspended in the solvent.

### Example 16

The suspension of example 15, wherein the plurality of surface-modified iron nitride nanoparticles have an average diameter of less than 100 nm.

### Example 17

The suspension of example 16, wherein the plurality of surface-modified iron nitride nanoparticles have an average diameter of less than 50 nm.

### Example 18

The suspension of any of examples 15 through 17, wherein the plurality of surface-modified iron nitride nanoparticles have a substantially unimodal size distribution.

### Example 19

The suspension of any of examples 15 through 18, wherein the plurality of surface-modified iron nitride nanoparticles are surface-modified by at least one of polyvinylpyrrolidone, a polyoxyalkylene amine derivative, polyethylene glycol, ethylene glycol monobutyl ether, nonylphenol, or tetramethylammonium hydroxide.

### Example 20

The suspension of any of examples 15 through 19, wherein the plurality of surface-modified iron nitride nanoparticles comprise at least one of γ′-Fe₄N, γ′-Fe₄NₓZ₁₋ₓ, α′-Fe₈N, α″-Fe₁₆NₓZ₂₋ₓ, or α′-Fe₈NₓZ₁₋ₓ, wherein Z comprises at least one of C, B, P, Si, or O.

### Example 21

A soft magnetic material includes a plurality of consolidated surface-modified iron nitride nanoparticles, wherein the plurality of consolidated surface-modified iron nitride nanoparticles have an average diameter less than about 100 nm.

### Example 22

The soft magnetic material of example 21, wherein the plurality of consolidated surface-modified iron nitride nanoparticles are dispersed in a binder.

### Example 23

The soft magnetic material of example 21 or 22, wherein the plurality of consolidated surface-modified iron nitride nanoparticles have a substantially irregular shape.

### Example 24

The soft magnetic material of any of examples 21 through 23, wherein the plurality of consolidated surface-modified iron nitride nanoparticles have a coercivity less than about 200 Oe.

### Example 25

The soft magnetic material of any of examples 21 through 24, wherein the plurality of consolidated surface-modified iron nitride nanoparticles are surface-modified by at least one of polyvinylpyrrolidone, a polyoxyalkylene amine derivative, polyethylene glycol, ethylene glycol monobutyl ether, nonylphenol, or tetramethylammonium hydroxide.

### Example 26

The soft magnetic material of any of examples 21 through 25, wherein the plurality of consolidated surface-modified iron nitride nanoparticles comprise at least one of γ′-Fe₄N, γ′-Fe₄NₓZ₁₋ₓ, α′-Fe₈N, α″-Fe₁₆NₓZ₂₋ₓ, or α′-Fe₈NₓZ₁₋ₓ, wherein Z comprises at least one of C, B, P, Si, or O.

Various examples have been described. These and other examples are within the scope of the following claims.

What is claimed is:

1. A method comprising:
wet ball milling a plurality of iron nitride nanoparticles in presence of a surface active agent to modify a surface of the plurality of iron nitride nanoparticles and form a plurality of surface-modified iron nitride nanoparticles.

2. The method of claim 1, wherein the plurality of iron nitride nanoparticles comprise at least one of γ′-Fe₄N, α′-Fe₈N, α″-Fe₁₆NₓZ₂₋ₓ, or α′-Fe₈NₓZ₁₋ₓ, wherein Z comprises at least one of C, B, P, Si, or O.

3. The method of claim 1, further comprising:
centrifuging the plurality of surface-modified iron nitride nanoparticles to separate a set of surface-modified iron nitride nanoparticles having a selected size profile from the plurality of surface-modified iron nitride nanoparticles.

4. The method of claim 1, further comprising:
forming the plurality of iron nitride nanoparticles from a plurality of iron oxide nanoparticles using a gas phase nitriding process.

5. The method of claim 4, wherein the plurality of iron oxide nanoparticles comprise a plurality of γ-Fe₂O₃ nanoparticles.

6. The method of claim 4, wherein the gas phase nitriding process comprises:
reducing, in a furnace, the plurality of iron oxide nanoparticles to form a plurality of iron nanoparticles; and
nitriding the plurality of iron particles to form the plurality of iron nitride nanoparticles.

7. The method of claim 6, wherein nitriding the plurality of iron particles to form the plurality of iron nitride nanoparticles further comprises forming the plurality of iron nitride nanoparticles comprising at least one of Fe₄N, Fe₄NₓZ₁₋ₓ, α″-Fe₁₆NₓZ₂₋ₓ, or α′-Fe₈NₓZ₁₋ₓ, where Z includes at least one of C, B, P, Si, or O based on introduction of at least one of carbon, boron, silicon, phosphorus, or oxygen to the furnace.

8. The method of claim 1, wherein wet ball milling the plurality of iron nitride nanoparticles in the presence of the surface active agent comprises wet ball milling the plurality of iron nitride nanoparticles in the presence of a source of at least one of: polyvinylpyrrolidone, a polyoxyalkylene amine derivative, polyethylene glycol, ethylene glycol monobutyl ether, nonylphenol, or tetramethylammonium hydroxide.

9. The method of claim 1, wherein wet ball milling the plurality of iron nitride nanoparticles in the presence of the surface active agent comprises wet ball milling for a period of between about 1 hour and about 10 hours at an rpm of between about 100 revolutions per minute (RPM) and about 1000 RPM.

10. The method of claim 3, wherein centrifuging the plurality of surface-modified iron nitride nanoparticles comprises centrifuging the plurality of surface-modified iron nitride nanoparticles at a revolutions per minute (RPM) and for a time selected to obtain a set of surface-modified iron nitride nanoparticles having at least one of an average diameter of less than about 100 nm or a substantially unimodal size distribution.

11. The method of claim 3, wherein centrifuging the plurality of surface-modified iron nitride nanoparticles comprises centrifuging the plurality of surface-modified iron nitride nanoparticles at an rpm of about 2,000 revolutions per minute (RPM) or about 11,000 RPM.

12. A suspension comprising:
a solvent; and
a plurality of surface-modified iron nitride nanoparticles suspended in the solvent.

13. The suspension of claim 12, wherein the plurality of surface-modified iron nitride nanoparticles have an average diameter of less than 100 nm.

14. The suspension of claim 13, wherein the plurality of surface-modified iron nitride nanoparticles have an average diameter of less than 20 nm.

15. The suspension of claim 12, wherein the plurality of surface-modified iron nitride nanoparticles have a substantially unimodal size distribution.

16. The suspension of claim 12, wherein the plurality of surface-modified iron nitride nanoparticles are surface-modified by at least one of polyvinylpyrrolidone, a polyoxyalkylene amine derivative, polyethylene glycol, ethylene glycol monobutyl ether, nonylphenol, or tetramethylammonium hydroxide.

17. The suspension of claim 12, wherein the plurality of surface-modified iron nitride nanoparticles comprise at least one of γ′-Fe₄N, γ′-Fe₄NₓZ₁₋ₓ, α′-Fe₈N, α″-Fe₁₆NₓZ₂₋ₓ, or α′-Fe₈NₓZ₁₋ₓ, wherein Z comprises at least one of C, B, P, Si, or O.

18. A soft magnetic material, comprising:
a plurality of consolidated surface-modified iron nitride nanoparticles,
wherein the plurality of consolidated surface-modified iron nitride nanoparticles have an average diameter of less than 100 nm.

19. The soft magnetic material of claim 18, wherein the plurality of consolidated surface-modified iron nitride nanoparticles have a substantially irregular shape.

20. The soft magnetic material of claim 18, wherein the plurality of consolidated surface-modified iron nitride nanoparticles are dispersed in a binder.
